(19) United States
(12) Patent Application Publication
(10) Pub. No.: US 2025/0232854 A1
(43) Pub. Date: Jul. 17, 2025

(54) Concept Mapping Using Large Language Models

(71) Applicant: Cerner Innovation, Inc., Kansas City, MO (US)

(72) Inventors: Suman Pal, Bangalore (IN); Pragnya Ranjan Pradhan, Similipada (IN); Oshin Benny Anto, Kerala (IN); Chetan KV, Bangalore (IN); Rupanjali Chaudhuri, Bangalore (IN); Vadim Khotilovich, Leawood, KS (US); Monica Gaur, Delhi (IN)

(73) Assignee: Cerner Innovation, Inc., Kansas City, MO (US)

(21) Appl. No.: 18/410,219

(22) Filed: Jan. 11, 2024

(51) Int. Cl. G16H 10/60; G06F 40/20

(52) U.S. Cl. CPC G16H 10/60; G06F 40/20

(57) ABSTRACT

Techniques for generating recommendations of candidate standard codes for association with unmapped proprietary codes are disclosed. Initially, the system generates vector embeddings for mapped standard codes by applying a vector embedding function to datasets of proprietary codes that are mapped to the respective mapped standard codes. The system generates vector embeddings for unmapped standard codes by applying a vector embedding function to a dataset of the unmapped standard codes. The system compares a target vector embedding for a target unmapped proprietary code to the vector embeddings computed for each of the mapped and unmapped standard codes. Based on a similarity measure between the target vector embedding and the vector embeddings for the mapped and unmapped standard codes, the system selects a subset of the mapped and unmapped standard codes for recommending to the user as a set of candidate standard codes for mapping to the target unmapped proprietary code.

### TECHNICAL FIELD

The present disclosure relates to mapping proprietary codes to standard codes. In particular, the present disclosure relates to using large language models for mapping proprietary codes to standard codes.

### BACKGROUND

The healthcare data present across multiple healthcare systems became connected and more accessible with the widespread adoption of Electronic Health Records (EHRs) by healthcare providers. EHRs have become an integral part of modern healthcare systems, offering several benefits over traditional paper-based records. EHRs are digital versions of a patient's medical history, including their diagnosis, treatments, medications, allergies, laboratory results, and other relevant healthcare information. This information may be presented as code values that are present under their respective type of field tables, known as code sets. A code set has a list of code values used to describe a specific purpose/intent. Healthcare data across various client domains are filled with ambiguous textual representations that may be present in the form of synonyms, acronyms, and abbreviations. This creates huge variance as the code values under various code sets are named differently though they have semantic equivalence.

The approaches described in this section are approaches that could be pursued but not necessarily approaches that have been previously conceived or pursued. Therefore, unless otherwise indicated, it should not be assumed that any of the approaches described in this section qualify as prior art merely by virtue of their inclusion in this section.

### BRIEF DESCRIPTION OF THE DRAWINGS

The embodiments are illustrated by way of example and not by way of limitation in the figures of the accompanying drawings. It should be noted that references to “an” or “one” embodiment in this disclosure are not necessarily to the same embodiment, and they mean at least one. In the drawings:

FIG. 1 illustrates a system in accordance with one or more embodiments;

FIGS. 2A-2C illustrate an example set of operations for a mapping target unmapped proprietary code to mapped or unmapped standard codes in accordance with one or more embodiments;

FIGS. 3A-3D illustrate an example of data flow during an example set of operations for presenting a recommendation of candidate mapped and unmapped standard codes for a target unmapped proprietary code;

FIG. 4 illustrates an interface for presenting recommendations of candidate standard codes for mapping to unmapped proprietary codes; and

FIG. 5 shows a block diagram that illustrates a computer system in accordance with one or more embodiments.

### DETAILED DESCRIPTION

In the following description, for the purposes of explanation, numerous specific details are set forth to provide a thorough understanding. One or more embodiments may be practiced without these specific details. Features described in one embodiment may be combined with features described in a different embodiment. In some examples, well-known structures and devices are described with reference to a block diagram form to avoid unnecessarily obscuring the present disclosure.
1. GENERAL OVERVIEW
2. EVENT CODE MAPPING SYSTEM
3. RECOMMENDING CANDIDATE MAPPED AND UNMAPPED STANDARD CODES FOR MAPPING TO A TARGET UNMAPPED PROPRIETARY CODE
4. EXAMPLE MAPPING OPERATIONS
5. RECOMMENDATION INTERFACE
6. HARDWARE OVERVIEW
7. MISCELLANEOUS; EXTENSIONS

### 1. General Overview

One or more embodiments generate recommendations of candidate mapped and unmapped standard codes for association with an unmapped proprietary code. Proprietary codes, as referred to herein, include reference codes particular to organizations or vendors. Standard codes, as referred to herein, are industry or standardized codes (LOINC, SNOMED, RxNorm). Mapping proprietary codes to standard codes enhances data interoperability and plays a crucial role in improving the overall quality of healthcare delivery and patient outcomes.

Initially, the system generates vector embeddings for mapped standard codes by applying a vector embedding function to datasets of proprietary codes that are mapped to the respective mapped standard codes. Applying a vector embedding function to the mapped standard codes includes applying the vector embedding function to attributes or textual descriptions of each of the proprietary codes that are mapped to the respective mapped standard codes. This may also include applying the vector embedding function to the attributes or textual descriptions of the respective mapped standard code.

The system generates vector embeddings for unmapped standard codes by applying a vector embedding function to a dataset of the unmapped standard codes. Applying a vector embedding function to the unmapped standard codes includes applying the vector embedding function to the attributes or textual descriptions of the unmapped standard codes.

In an embodiment, the system compares a target vector embedding for a target unmapped proprietary code to the vector embeddings computed for each of the mapped and unmapped standard codes. Based on a similarity measure between the target vector embedding and the vector embeddings for the mapped and unmapped standard codes, the mapped and unmapped standard codes are ranked. The system selects a subset of the mapped and unmapped standard codes for recommending to the user as a set of candidate standard codes for mapping to the target unmapped proprietary code. Upon receipt of user input selecting a particular standard code of the set of candidate mapped and unmapped standard codes, the system stores an association, or mapping, between the particular standard code and the target unmapped proprietary code.

One or more embodiments described in this Specification and/or recited in the claims may not be included in this General Overview section.

### 2. Event Code Mapping System

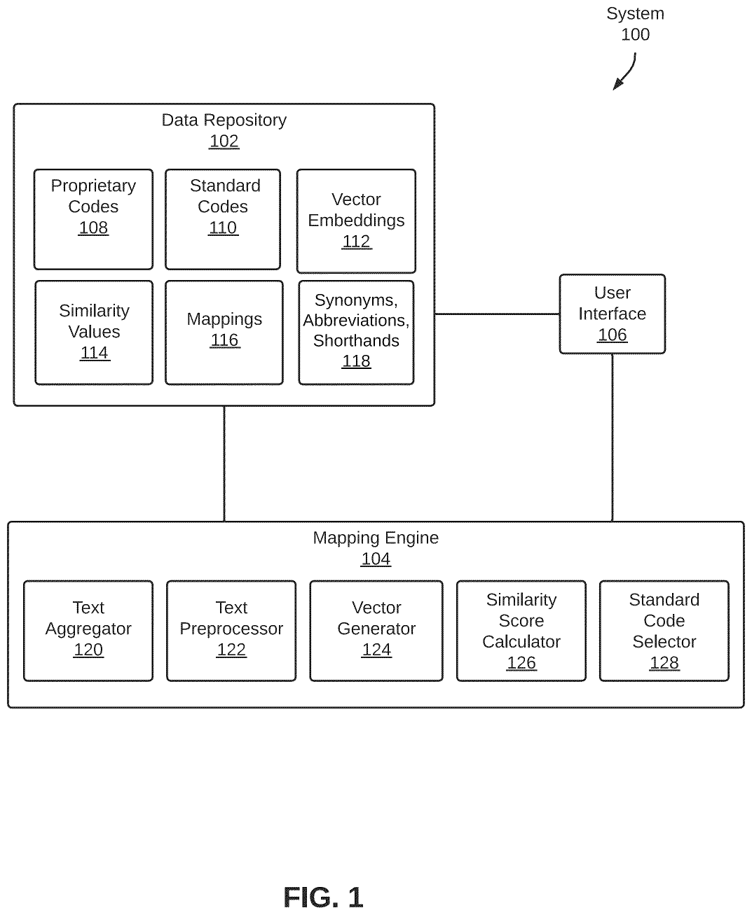

FIG. 1 illustrates a mapping system 100 in accordance with one or more embodiments. As illustrated in FIG. 1, the system 100 includes a data repository 102, a mapping engine 104, and a user interface 106. In one or more embodiments, the system 100 may include more or fewer components than the components illustrated in FIG. 1. The components illustrated in FIG. 1 may be local to or remote from each other. The components illustrated in FIG. 1 may be implemented in software and/or hardware. Each component may be distributed over multiple applications and/or machines. Multiple components may be combined into one application and/or machine. Operations described with respect to one component may instead be performed by another component.

In one or more embodiments, a data repository 102 is any type of storage unit and/or device (e.g., a file system, database, collection of tables, or any other storage mechanism) for storing data. Further, a data repository 102 may include multiple different storage units and/or devices. The multiple different storage units and/or devices may or may not be of the same type or located at the same physical site. Further, a data repository 102 may be implemented or executed on the same computing system as the mapping engine 104 and the user interface 106. Alternatively, or additionally, a data repository 102 may be implemented or executed on a computing system separate from the mapping engine 104 and the user interface 106. The data repository 102 may be communicatively coupled to the mapping engine 104 and the user interface 106 via a direct connection or via a network.

In embodiments, the data repository 102 is populated with information from a variety of sources and/or systems. The data repository 102 may be populated with data such as proprietary codes 108, standard codes 110, vector embeddings 116, similarity values 118, mappings 120, and synonyms, abbreviations, and shorthands 118. Any of this information may be stored in a structured format (e.g., a table).

In one or more embodiments, proprietary codes 108 are reference codes for clinical events and/or non-clinical events that are customized for consumers. When creating proprietary codes 108, local practice may be favored over uniformity of content, resulting in different consumers having unique sets of proprietary codes 108. Although the names of the proprietary codes 108 may differ between consumers, many of the proprietary codes 108 have semantic equivalences. Mapped proprietary codes are proprietary codes that have been mapped to a standard code, e.g., LOINC, SNOMED, RXNorm. Unmapped proprietary codes are codes that have not been mapped to a standard code.

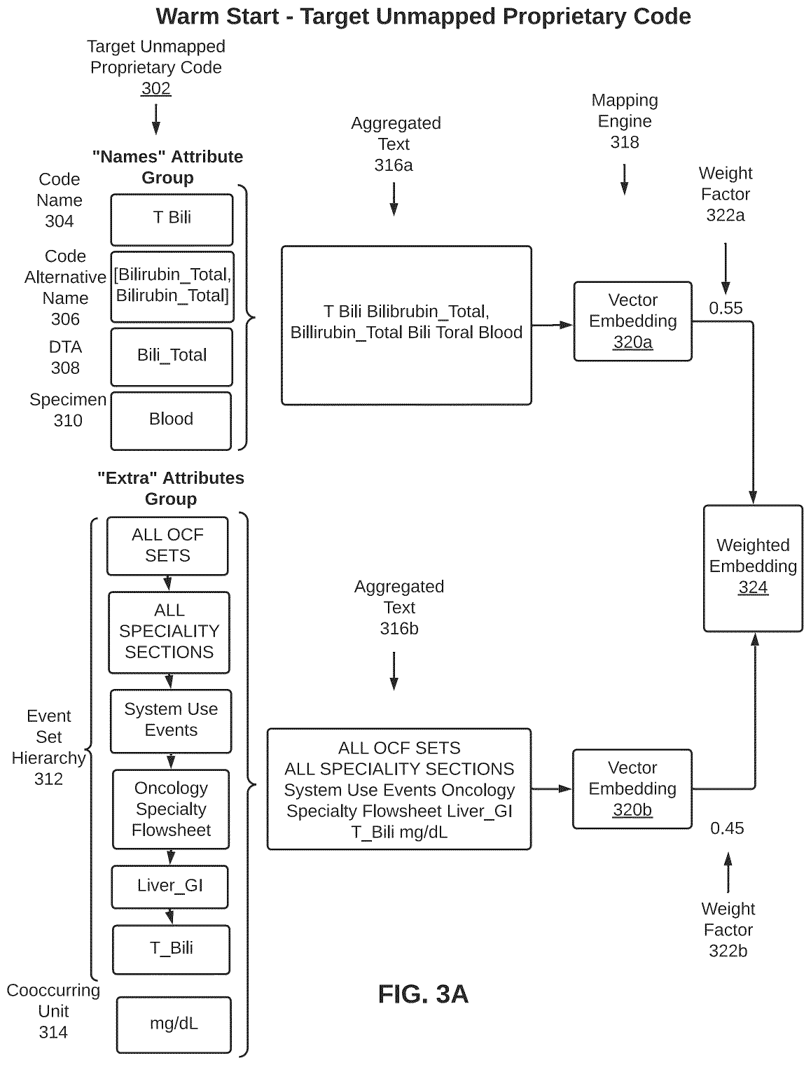

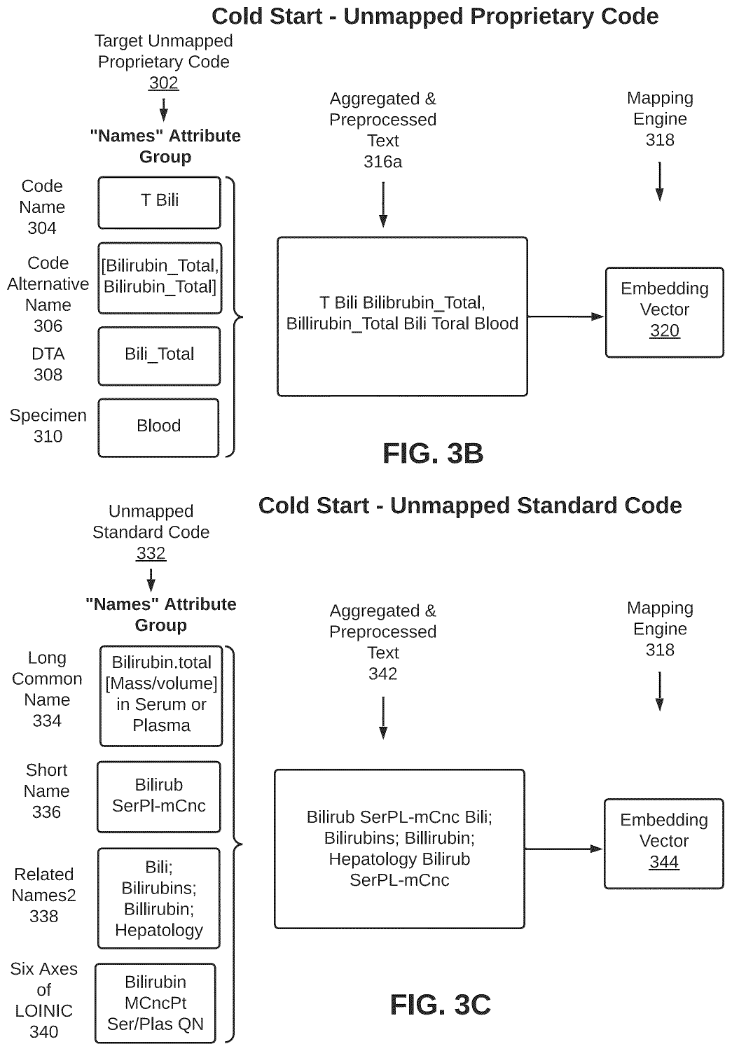

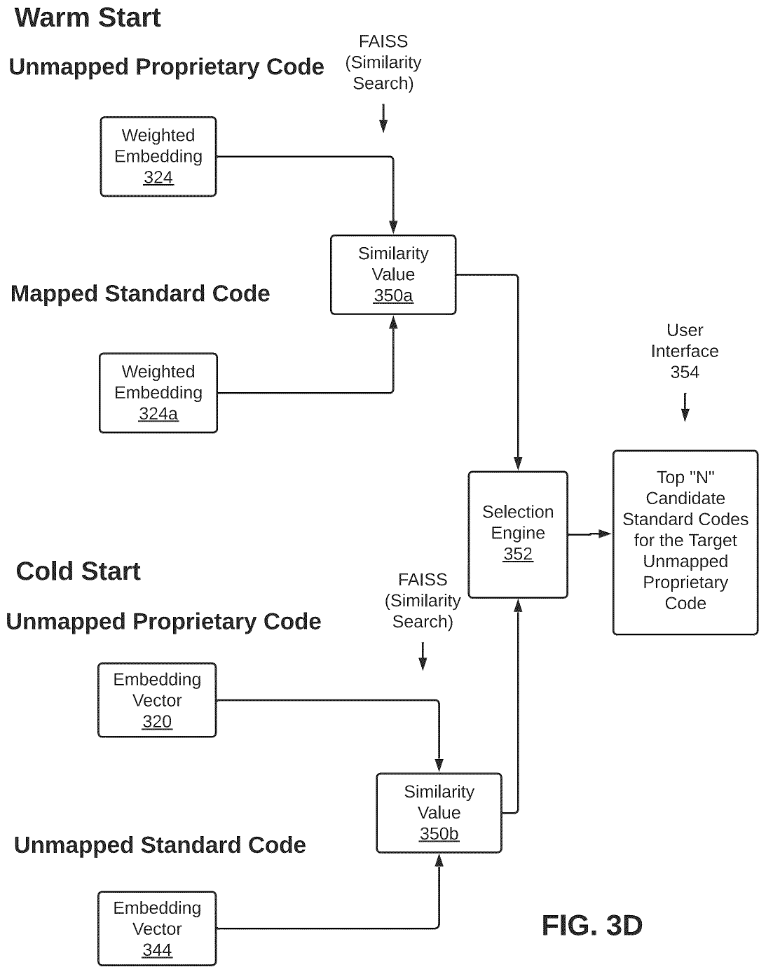

In embodiments, proprietary codes 108 include attributes or variables, i.e., reference data, for identifying clinical and/or non-clinical events. The proprietary codes 108, mapped and unmapped, may be sourced from one or more disparate consumer databases. The attributes for each of the proprietary codes 108 may be sorted into groups, e.g., a “Names” attribute group and an “Extras” attribute group. The “Names” attribute group may include consumer specific codes, descriptions, identifies, and/or unit measurement types. For example, as shown in FIG. 3A, the “Names” attribute group includes Code Name, Code Alternate Name, DTA (Discrete Task Assay), and Specimen. The “Extras” attribute group may include an event set hierarchy and/or additional reference data. An event set hierarchy is a hierarchical or parent/child relationship of events sets. The additional reference data may include a cooccurring unit. Co-occurring units are associated units to the value for the event code data collected.

In some embodiments, the proprietary codes 108 include Code Set 72. Code Set 72, also known as Cerner Clinical Event Codes, is a proprietary code set maintained by Cerner Corporation. Code Set 72 is an extensive collection of codes used to represent various clinical and non-clinical events, including clinical documents, note types, immunizations, and clinical observations, such as laboratory results and vital signs. Code Set 72 is highly customized by Cerner clients, and the specific codes used may vary depending on the client's healthcare system. The general structure and purpose of the code set remain consistent across Cerner clients. Code Set 72 is a very large code set, encompassing a wide range of clinical events. The specific codes used in Code Set 72 are tailored to meet the specific needs of each Cerner client.

In embodiments, the standard codes 110 are a set of industry or standardized codes that are widely adopted and used across the healthcare industry. Standard codes 110 represent various aspects of patient care, procedures, diagnoses, and other healthcare-related information. Example standard codes include ICD-10 (International Classification of Diseases, 10th Edition), CPT (Current Procedural Terminology), HCPCS (Healthcare Common Procedure Coding System), SNOMED CT (Systematized Nomenclature of Medicine Clinical Terms), NDC (National Drug Code), and RxNorm. A standard code may be mapped to multiple proprietary codes.

In some embodiments, the standard codes 108 are Logical Observation Identifiers Names and Codes (LOINC®). LOINC is a universal standard for identifying health measurements, observations, and documents. LOINC is a common language that allows different healthcare systems to exchange data seamlessly. LOINC codes are used to represent the “question” for a test or measurement, such as “blood glucose” or “body mass index,” to aid in ensuring that the results of tests and measurements are interpreted accurately and consistently across different systems. The LOINC database contains over 90,000 codes that are translated into more than 40 languages. LOINC is used by a wide variety of organizations, including hospitals, clinics, laboratories, and government agencies. LOINC helps to ensure that data can be exchanged seamlessly between different healthcare systems, thereby improving patient care by making it easier for clinicians to access and understand patient data. LOINC codes are unique and unambiguous, this helps to reduce errors in data entry and interpretation. LOINC can be used to link data from different sources, improving research on a variety of health topics.

In embodiments, standard codes 110 include attributes or variables, i.e., reference data, for identifying clinical and/or non-clinical events. Similar to the proprietary codes 108, the attributes for each of the standard codes 110 may be sorted into groups. A “Names” attribute group may include code names, code references, and/or observations. For example, as shown in FIG. 3C, the “Names” attribute group for a LOINC code includes Long Common Name, Short Name, Related Names 2, and Six axes of LOINC. Long Common Names are designed to be the user-friendly representation of a LOINC term, providing a human-readable format for understanding the meaning of a LOINC code. The Related Names 2 are synonyms that are associated with the specific LOINC code.

The Six axes of LOINC include component, property, time, system, scale, and method. The component axis represents the analyte or property being measured. The component axis describes what is being observed or measured, such as glucose, cholesterol, or blood pressure. The property axis describes the characteristics of the analyte or property. The property axis provides additional information about the type of measurement being made, such as mass, concentration, or time. The time axis specifies the timing of the observation, indicating when the measurement was taken or how the observation is related to time. For example, the time axis might indicate whether the observation is a point in time, a 24-hour urine collection, or a fasting specimen. The system axis specifies the system or specimen source from where the observation is derived. The system axis provides information about the origin of the specimen, such as blood, urine, or cerebrospinal fluid. The scale axis describes the scale of measurement for the observation, such as qualitative, ordinal, or quantitative. The scale axis provides information about how the observation is expressed numerically or categorically. The method axis represents the procedure or method used to perform the observation. The method axis provides details about the specific technique, instrument, or protocol used to obtain the result.

In one or more embodiments, the vector embeddings 112 in the data repository 102 are text that have been converted to a numeric format. The vector embeddings 112 are representations of individual words for text analysis, typically in the form of a real-valued vector. The vector embeddings 112 may represent individual text or may represent an aggregation of text. As will be described in further detail below with respect to mapping engine 104, the vector embeddings 112 may be formed using various word embedding techniques. The vector embeddings 112 represent mapped and unmapped standard codes and unmapped proprietary codes.

In embodiments, the similarity values or measures 114 in the data repository 102 provide an indication of the similarity between the vector embeddings 112 of a standard code 110, mapped or unmapped, and unmapped proprietary codes. The higher the similarity values 114, i.e., the closer to 1.0, the greater a semantic match between the vector embeddings 112. The similarity values 114 may each be assigned a ranking category. For example, a similarity value less than 0.90 may be categorized as “low”; a similarity value equal to or greater than 0.90 and less than 0.98 may be categorized as “medium”; and a similarity value greater than or equal to 0.98 may be categorized as “high”. The similarity values 114 may be weighted to reflect the relevance of the type of data used to calculate the vector embeddings. For example, data with a high relevance to determining an appropriate mapping of a proprietary code may receive a weight of 0.55, while data with less relevance to the mapping may receive a weight of 0.45.

In embodiments, mappings 116 include mappings between proprietary codes 108 and standard codes 110. When a mapped standard code is mapped to the unmapped proprietary code, the unmapped proprietary code provides a dataset for the mapped standard code that is for future charting. When an unmapped standard code is mapped to a proprietary code 108, the unmapped standard code becomes a mapped standard code. Multiple proprietary codes may be mapped to an individual standard code.

In some embodiments, the synonyms, abbreviations, and shorthands 118 are included in a table that provides synonyms, abbreviations, and/or shorthands that may or may not be specific to a consumer and corresponding expansions for the respective synonym, abbreviation or shorthand. For example, “SBP” may correspond to “systolic blood pressure”; “LMP” may correspond to “last menstrual period”; “I:E” may correspond to “inspiratory to expiratory ratio”; and “GAD7” may correspond to “general anxiety disorder”.

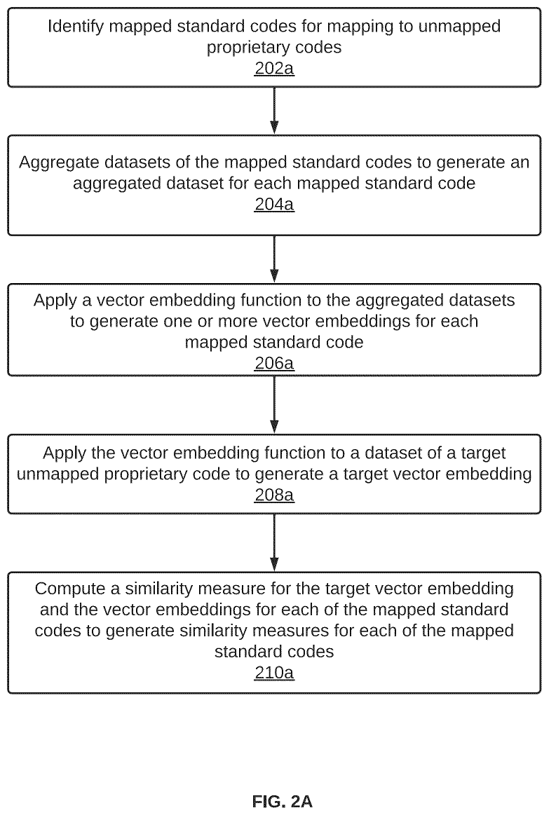

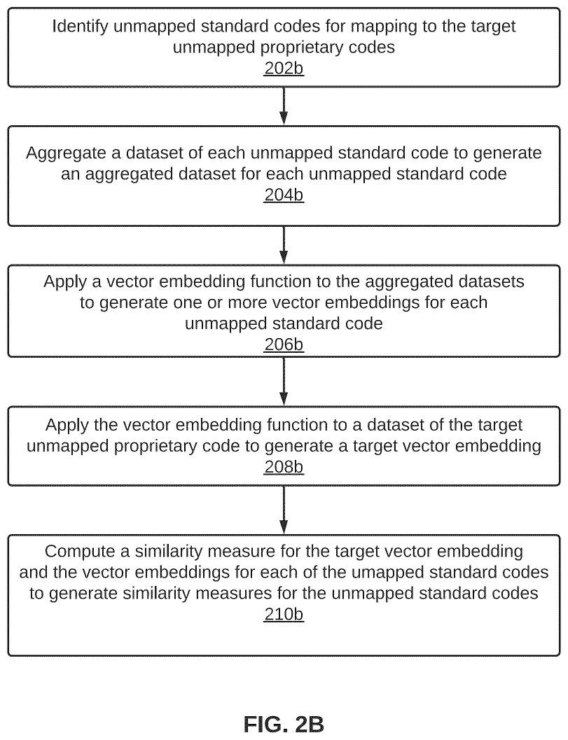

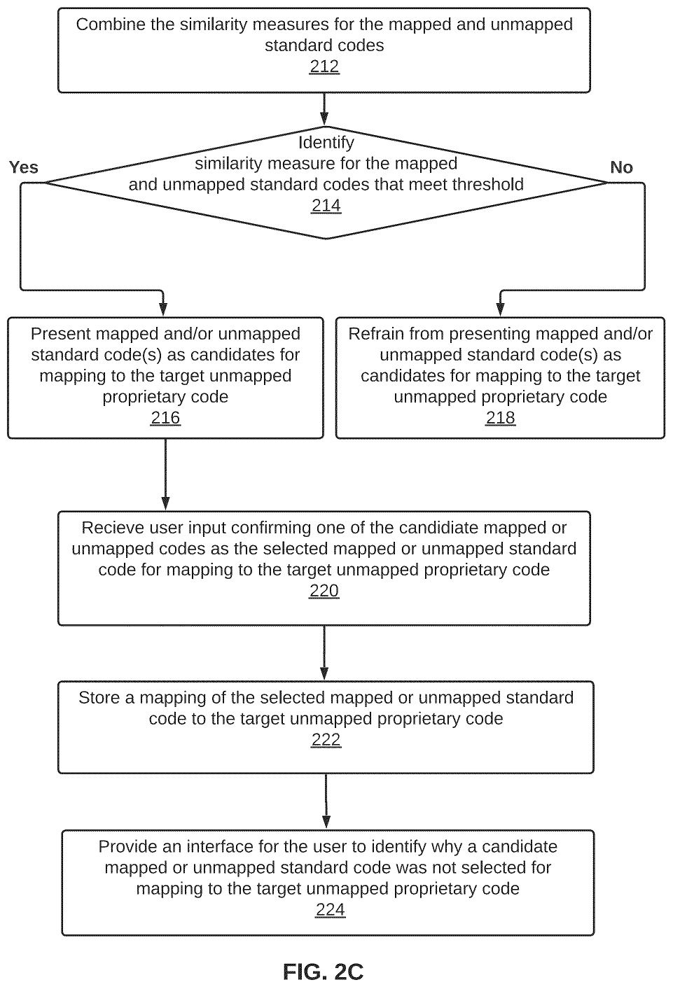

In embodiments, the mapping engine 104 of the system 100 is hardware and/or software configured to map unmapped proprietary codes to mapped and unmapped standard codes. Examples of operations for providing recommendations of candidate mapped and unmapped standard codes are described below with references to FIG. 2A-2C. The mapping engine 104 may include a text aggregator 120, a text preprocessor 122, a vector generator 124, a similarity score calculator 126, and a standard code selector 128.

In one or more embodiments, the text aggregator 120 aggregates text from the attributes of the proprietary codes 108 and the attributes of the standard codes 110. The text aggregator 120 may aggregate text prior to preprocessing of the text by the text preprocessor 122 or after preprocessing of the text.

In some embodiments, the text is processed by the text preprocessor 122 prior to applying the vector generator 124 to the aggregated text to generate vector embeddings 112. The text preprocessor may perform functions, such as converting the text into lower case and/or retaining numeric tokens. Text is converted to lower case to provide uniformity to the text. In prior art mapping engines, numeric tokens are typically removed during text preprocessing. Removal of numeric tokens may eliminate a distinguishing feature of a concept. For example, “Right Ear 500 Hz POC” and “Right Ear 1000 Hz POC” are differentiated using a numeric token. By retaining numeric tokens, misclassifications are more readily avoided.

In embodiments, text preprocessing may further include handling special characters, removing unwanted text, and custom preprocessing. Handling special characters includes addressing symbols and special characters. For example, text line “D-Dimer” requires special attention. Replacing the “-” with a blank space creates two different tokens, namely “D” and “Dimer”. As such, using traditional text preprocessing, the entire context of “D-Dimer” is lost. By addressing special characters, the context of the terms is maintained. Removing unwanted text from the event set hierarchy includes removing text that is present in all event set hierarchy data. Specifically, there are core event sets that are present in all event set hierarchy data. Since the core event sets do not add any new information between datasets, the core event sets are removed from the data. Custom preprocessing includes attending to consumer specific text such as synonyms, abbreviations, and shorthands. The custom preprocessing may consult the synonyms, abbreviations, and shorthands 118 stored in the data repository 102 to provide expansions for various consumer specific synonyms, abbreviations, and shorthands.

In some embodiments, the vector generator 124 includes software and/or hardware for performing one or more vector embedding functions. Vector embedding functions are mathematical functions that map objects, such as words, sentences, or other data points, into vector representations in a multi-dimensional space. These vector representations are used to capture the semantic or contextual meaning of the objects in a numerical format that can be easily processed by machine learning algorithms.

In some embodiments, the vector embedding functions are word embedding techniques. Word embedding techniques use natural language processing (NLP) and machine learning to represent words as dense vectors of real numbers. Word embedding techniques aim to capture the semantic and syntactic meaning of words as well as their relationships with other words in a language. Word embedding techniques include Term Frequency-Inverse Document Frequency (TF-IDF), Word2Vec, Global Vectors (GLOVE), Large Language Models (LLM), and BioWordVec fastText.

Each of these word embedding techniques includes salient features. The TF-IDF model is designed to give more weight to the words that are very specific to certain documents but give less weight to the words that are more general and occur across most documents. The Word2Vec model represents words in the form of dense vectors by capturing syntactic (grammar) and semantic (meaning) relationships. Given a large enough dataset, the Word2vec model provides strong estimates about a word's meaning based on its frequency of occurrence in the text. The GLOVE model is an unsupervised learning model that can be used to obtain dense word vectors like the Word2Vec model. The GLOVE model first creates a large word-context, co-occurrence matrix consisting of pairs (word, context). Each element in this matrix represents how often a word or a sequence of words occurs within the context and then applies matrix factorization to approximate this matrix. The BioWordVec fastText model is 200-dimensional word embeddings trained on PubMed and MIMIC-III data and is the extension of the original BioWord Vec that provides fastText word embeddings trained using PubMed and MeSH. A subword embedding model used by the BioWordVec fastText model better handles Out of Vocabulary (OOV) tokens and improves the quality of the word embeddings.

In one or more embodiments, the word embedding techniques include Self-Alignment Pretraining for Biomedical Entity Representations (SAPBERT). The SAPBERT model leverages the Unified Medical Language System (UMLS), a comprehensive resource in the biomedical field. UMLS incorporates a vast collection of biomedical concepts and synonyms from various controlled vocabularies like MeSH, SNOMEDCT, RxNorm, Gene Ontology, and OMIM. This rich source of data greatly enhances the model's understanding of medical terminology and relationships. SAPBERT provides contextual embeddings, meaning that it can understand the meaning of words and phrases in context. This is crucial for understanding complex medical texts and making accurate predictions in healthcare applications. The SAPBERT model can accurately capture fine-grained semantic relationships and heterogeneous naming in the biomedical domain compared to other variants of BERT. The ability of SAPBERT to handle out-of-vocabulary (OOV) terms, misspelled words, and rare medical terms provides a significant advantage over other models.

In embodiments, the similarity score calculator 126 calculates a similarity between vector embeddings for standard codes and vector embeddings for unmapped proprietary codes. The similarity score calculator 126 may include the Facebook AI Similarity Search (FAISS). FAISS is an open-source library developed by Facebook for efficient similarity search and clustering of high-dimensional vectors. FAISS is optimized for both CPU and GPU architectures, enabling fast and scalable similarity search operations on large datasets. FAISS supports a range of similarity metrics, including Euclidean distance, cosine similarity, inner product, and L2 distance. FAISS offers various indexing methods, including the inverted file, Hierarchical Navigable Small World (HNSW), and product quantization. HNSW is an algorithm for efficient similarity search in high-dimensional spaces. These indexing techniques help speed up nearest-neighbor searches in high-dimensional spaces. In an embodiment, FAISS is combined with HNSW as the indexing approach. FAISS can be integrated with popular machine learning libraries and frameworks, such as PyTorch and TensorFlow, making it easier to incorporate similarity searches into machine learning pipelines. This may lead to significant improvements in the speed and scalability of the similarity search operations. As an open-source library, FAISS is available for developers and researchers to use, modify, and contribute to its development.

In one or more embodiments, recommendations for an unmapped proprietary code is provided by the standard code selector 128. The standard code selector 128 presents candidate mapped and unmapped standard codes to the user interface 106 based on the similarity values 114 provided by the similarity score calculator 126. The standard code selector 128 may present an “N” number of candidate standard codes ranked by the similarity values between the vector embeddings of the candidate standard codes and the vector embedding of the target unmapped proprietary code. Alternatively, the standard code selector 128 may present every candidate standard code having a similarity measure with the unmapped proprietary code above a threshold.

In some embodiments, the standard code selector 128 provides recommendations of one or more candidate unmapped proprietary codes for each standard code. The candidate unmapped proprietary codes may be presented in any of the same manners as described above with respect to the candidate standard codes.

In an embodiment, the mapping engine 104 is implemented on one or more digital devices. The term “digital device” generally refers to any hardware device that includes a processor. A digital device may refer to a physical device executing an application or a virtual machine. Examples of digital devices include a computer, a tablet, a laptop, a desktop, a netbook, a server, a web server, a network policy server, a proxy server, a generic machine, a function-specific hardware device, a hardware router, a hardware switch, a hardware firewall, a hardware firewall, a hardware network address translator (NAT), a hardware load balancer, a mainframe, a television, a content receiver, a set-top box, a printer, a mobile handset, a smartphone, a personal digital assistant (PDA), a wireless receiver and/or transmitter, a base station, a communication management device, a router, a switch, a controller, an access point, and/or a client device.

In one or more embodiments, user interface 106 refers to hardware and/or software configured to facilitate communications between a user and mapping engine 104. User interface 106 renders user interface elements and receives input via user interface elements. Examples of interfaces include a graphical user interface (GUI), a command line interface (CLI), a haptic interface, and a voice command interface. Examples of user interface elements include checkboxes, radio buttons, dropdown lists, list boxes, buttons, toggles, text fields, date and time selectors, command lines, sliders, pages, and forms.

In an embodiment, different components of user interface 106 are specified in different languages. The behavior of user interface elements is specified in a dynamic programming language, such as JavaScript. The content of user interface elements is specified in a markup language, such as hypertext markup language (HTML) or XML User Interface Language (XUL). The layout of user interface elements is specified in a style sheet language, such as Cascading Style Sheets (CSS). Alternatively, user interface 106 is specified in one or more other languages, such as Java, C, or C++.

### 3. Recommending Candidate Mapped and Unmapped Standard Codes for Mapping to a Target Unmapped Proprietary Code

FIGS. 2A-2C illustrate an example set of operations for recommending candidate standard codes for mapping to unmapped proprietary codes in accordance with one or more embodiments. One or more operations illustrated in FIGS. 2A-2C may be modified, rearranged, or omitted. Accordingly, the particular sequence of operations illustrated in FIG. 2A-2C should not be construed as limiting the scope of one or more embodiments.

The mapping operations includes providing one or more mapped and/or unmapped standard codes as candidates for unmapped proprietary codes. Operations using mapped standard codes, i.e., standard codes that are mapped to one or more proprietary codes, are referred to herein as a warm start. Operations using unmapped standard codes, i.e., standard codes that are not yet mapped to one or more proprietary codes, are referred to herein as a cold start.

In the warm start, illustrated in FIG. 2A, one or more embodiments identify mapped standard codes for mapping to unmapped proprietary (Operation 202a). Standard codes are industry or standardized codes that represent a clinical concept. Mapped standard codes are standard codes that have been mapped to one or more proprietary codes. Proprietary codes are reference or local codes for clinical and non-clinical events that are customized for consumers. Unmapped proprietary codes are proprietary codes that have not yet been mapped to a standard code.

One or more embodiments aggregate datasets of the mapped standard codes to generate an aggregated dataset for each mapped standard code (Operation 204a). The datasets for each of the mapped standard codes includes the datasets for each of the one or more proprietary codes mapped to the respective mapped standard code. The datasets for the one or more proprietary codes include attributes. The attributes include reference data for each of the proprietary codes. The attributes may be sorted into groups. The attribute groups may be aggregated individually or together. The datasets for each of the mapped standard codes may also include the attributes for the respective mapped standard code. In an example, the datasets include a “Names” attribute group and an “Extras” attribute group.

One or more embodiments apply a vector embedding function to the aggregated datasets to generate one or more vector embeddings for each mapped standard code (Operation 206a). The vector embedding function generates a vector embedding for each of the mapped standard codes. The vector embeddings are numerical representations of the aggregated text. The vector embedding function may include Self-Alignment Pretraining for Biomedical Entity Representations (SAPBERT).

In one or more embodiments, the vector embedding generated for the dataset of each of the mapped standard codes may be a weighted average of the vector embedding for each of the groups of attributes. For example, a weight applied to a first group of attributes, e.g., “Names”, is 0.55, and a weight applied to a second group of attributes, e.g., “Extras”, is 0.45. A grid-search approach may be used to determine the best weight.

One or more embodiments apply the vector embedding function to a dataset of a target unmapped proprietary code to generate a target vector embedding (Operation 208a). The vector embedding function generates the target vector embedding for the target unmapped proprietary code. The dataset of the target unmapped proprietary code include aggregated text from attributes of the target unmapped proprietary code. The attributes may be separated into groups. The groups of attributes may be aggregated separately or together. The vector embedding generated for the dataset of the target unmapped proprietary code may be a weighted average of the vector embedding for each of the groups of attributes.

One or more embodiments compute a similarity measure for the target vector embedding and vector embeddings for each of the mapped standard codes to generate similarity measures for each of the mapped standard codes (Operation 210a). The similarity values are a semantic similarity between the target vector embedding and the vector embeddings for each of the mapped standard codes.

In some embodiments, the similarity measures are calculated using Facebook AI Similarity Search (FAISS). FAISS may be combined with Hierarchical Navigable Small World (HNSW) as the indexing approach. Other indexing approaches may include Inverted File (IVF), Product Quantization (PQ), Locality Sensitive Hashing (LSH), and combinations of these approaches.

In the cold start, illustrated in FIG. 2B, one or more embodiments identify unmapped standard codes for mapping to the target unmapped proprietary codes (Operation 202b). The unmapped standard codes are industry or standardized codes that are not mapped to one or more proprietary codes.

One or more embodiments aggregate a dataset of the unmapped standard codes to generate an aggregated dataset for each unmapped standard code (Operation 204b). The dataset for each of the unmapped standard codes includes the attributes for the respective unmapped standard code. The dataset may include attributes from one or more groups of attributes for each of the respective unmapped standard codes.

One or more embodiments apply a vector embedding function to the aggregated datasets to generate one or more vector embeddings for each unmapped standard code (Operation 206b). The vector embedding function generates a vector embedding for each of the unmapped standard codes.

One or more embodiments apply the vector embedding function to a dataset of the target unmapped proprietary code to generate a target vector embedding (Operation 208b). The dataset of the target unmapped proprietary code may be the same or different from the dataset of the target unmapped proprietary code used in the warm start. For example, the dataset for the target unmapped proprietary code may be limited to a selection of the attributes used in the warm start.

One or more embodiments compute a similarity measure for the target vector embedding and the vector embeddings for each of the unmapped standard codes to generate similarity measures for the unmapped standard codes (Operation 210b). The similarity values are a semantic similarity between the target vector embedding and the vector embeddings for each of the unmapped standard codes. The similarity values for the unmapped standard codes may be computed in the same manner as the similarity values for the mapped standard codes, as described above.

With reference to the operations illustrated in FIG. 2C, one or more embodiments combine the similarity measures for the mapped and unmapped standard codes (Operation 212). The similarity measures for the mapped standard codes and the similarity measures for the unmapped standard codes are combined. The similarity measures may be ranked based on the computed similarity scores for each of the mapped and unmapped standard codes.

One or more embodiments identify similarity measures for the mapped and unmapped standard codes that meet a threshold (Operation 214). A threshold similarity measure may include a similarity measure above a predetermined measure, e.g., above 0.90.

One or more embodiments present mapped and/or unmapped standard codes as candidates for mapping to the target unmapped proprietary code (Operation 216). The mapped and/or unmapped standard codes presented as candidates for mapping to the target unmapped proprietary code include mapped and/or unmapped standard codes with a similarity measure above the threshold. Alternatively, the mapped and/or unmapped standard codes presented as candidates for mapping to the target unmapped proprietary code include a top “N” number of mapped and/or unmapped standard codes based on their similarity measure. The mapped and/or unmapped standard codes presented as candidates may be presented on an interface.

One or more embodiments refrain from presenting mapped and/or unmapped standard codes as candidates for mapping to the target unmapped proprietary code (Operation 218). The system does not present, as candidates for mapping to the target proprietary code, the mapped and/or unmapped standard codes that have a similarity measure below the threshold. Alternatively, the system does not present as candidates the mapped and/or unmapped standard codes that are outside the top “N” number of mapped and/or unmapped standard codes based on their similarity measure.

One or more embodiments receive user input confirming the candidate mapped or unmapped codes as the selected mapped or unmapped standard code for mapping to the target unmapped proprietary code (Operation 220). The user input may include selecting an icon representing the desired candidate mapped or unmapped standard code. The system may provide indication of a preferred candidate.

One or more embodiments store a mapping of the selected mapped or unmapped standard code to the target unmapped proprietary code (Operation 222). The mapping of the selected mapped or unmapped standard code to the target unmapped proprietary code may be used in subsequent mappings of the selected mapped or unmapped standard code to other unmapped proprietary codes. The mapping of the target unmapped proprietary code to the selected mapped or unmapped standard code provides an additional dataset for the selected mapped or unmapped standard code. The additional dataset for the selected mapped or unmapped standard code increases the accuracy and precision of future recommendations.

One or more embodiments provide an interface for the user to identify why a candidate mapped or unmapped standard code was not selected for mapping to the target unmapped proprietary code (Operation 224). To better understand why the user did not select a particular candidate mapped or unmapped standard code, the user may be prompted to identify why a candidate mapped or unmapped proprietary code was not selected. The user prompt may include an assortment of predefined user selectable responses and/or an input box for text entry.

In some embodiments, the vector embedding function includes a machine learning model. The machine learning model is trained on training datasets to compute vector embeddings from mapped and unmapped standard codes. Particular training data, of the training datasets, may include one or more historical mapped standard codes as well as a vector embedding corresponding to the historical mapped and unmapped standard codes. Applying the vector embedding function to the dataset of the target unmapped proprietary code includes applying the machine learning model to the dataset of the target unmapped proprietary code, receiving feedback based on an accuracy of results generated by applying the vector embedding function, and retraining the machine learning model based on the feedback.

### 4. Example Mapping Operations

A detailed example is described below for purposes of clarity. Components and/or operations described below should be understood as one specific example that may not be applicable to certain embodiments. Accordingly, components and/or operations described below should not be construed as limiting the scope of any of the claims.

FIG. 3A illustrates operations for generating weighted vector embeddings for a target unmapped proprietary code in a warm start. Initially, attributes for an unmapped proprietary code 302 are targeted. The attributes include a first group of attributes identified as “Names” and a second group of attributes identified as “Extras”. The “Names” attribute group includes Code Name 304, Code Alternative Name 306, Discrete Task Assay (DTA) 308, and Specimen 310. The “Extras” attribute group includes an Event Set Hierarchy 312 and Cooccurring Unit 314. Although shown including an entry in each of the data fields, it is understood that not every data field may include an entry.

The text from the data fields of the attributes for the target unmapped proprietary code 302 are aggregated to form text aggregates. More particularly, the text for the attributes in the “Names” attribute group, i.e., Code Name 304, Code Alternative Name 306, DTA 308, and Specimen 310 are combined into a first aggregate text 316a. The text for the attributes in the “Extra” attribute group, i.e., Event Set Hierarchy 312 and the Coocurring Unit 314, are combined into a second text aggregate 316b. The text may be preprocessed prior or subsequent to the aggregation of the text to provide uniformity and to address special characters, synonyms, abbreviations, and shorthands.

Upon completion of the text aggregation and preprocessing, a mapping engine 318 generates embedding vectors 320a, 320b for the respective first and second aggregated texts 316a, 316b. The embedding vectors 320a, 320b may be of individual tokens within the respective first and second aggregated texts 316a, 316b or of the entirety of the respective first and second aggregated texts 316a, 316b. The embedding vectors 320a, 320b are generated using a natural language process embedding model, e.g., SAPBERT.

Based on a grid-search approach, it was determined that the attributes in the “Names” attribute group contain more relevant information for determining candidate standard codes. Hence, a weight factor 322b of 0.55 is applied to the embedding vector 320a for the “Names” attribute group. A weight factor 322b of 0.45 is applied to the embedding vector 320b for the “Extras” attribute group. The weight factors 322a, 322b are applied to the respective individual embedding vectors 320a, 320b of the first and second aggregated texts 316a, 316b, respectively, to generate a first weighted vector embedding (not shown) and second weighted vector embedding (not shown); these are then combined to create a weighted embedding 324.

The operations (not shown) for generating a weighted embedding 324a (FIG. 3D) for mapped standard codes, e.g., LOINC codes, in the warm start are similar to the operations for generating the weighted vector embeddings 324 for the target unmapped proprietary code 302 described above with reference to FIG. 3A. More particularly, the “Names” attribute group includes the Code Name 304, Code Alternate Name 306, DTA 308, and Specimen 310 for each of the proprietary codes mapped to the respective mapped standard code. Similarly, the “Extras” attribute group includes the Event Set Hierarchy and Coocurring Unit for each of the proprietary codes mapped to the respective mapped standard code. In addition, the “Names” attribute group may include attributes for the mapped standard code. These may include Long Common Name 334 (FIG. 3C), Short Name 336, Related Names2 338, and the Six Axes of LOINC 340.

The text for each of the “Names” attribute group and the “Extras” attribute group are aggregated and preprocessed in the same manner as these attribute groups in the warm start for the target unmapped proprietary code. The aggregated and preprocessed texts (not shown) for the mapped standard codes are used to generate the embedding vectors (not shown) for the mapped standard codes as described above. Weight factors are applied to the respective individual embedding vectors for the aggregated and preprocessed text for the respective “Names” attribute group and the “Extras” attribute group to generate weighted vector embeddings (not shown); these are then combined to create a weighted embedding 344 (FIG. 3D).

FIG. 3B illustrates operations for generating the embedding vector 320a for the unmapped proprietary code 302 in a cold start. The operations for generating the embedding vector 320a for the unmapped proprietary code 302 are similar to the operations for generating the weighted embedding vector 324 for the unmapped proprietary code 302 described above with reference to FIG. 3A. Unlike in the warm start, generating the embedding vector 320a for the unmapped proprietary code 302 in the cold start, as shown, is limited to the attributes in the “Names” attribute group. In this manner, there is no weighting of the embedding vector 320a to create a weighted vector embedding. It is envisioned that the “Extras” attribute group may also be aggregated and preprocessed.

FIG. 3C illustrates operations for generating an embedding vector 340 for an unmapped standard code 332 in the cold start. The operations for generating the embedding vector 340 for the unmapped standard code 332 are similar to the operations for generating the embedding vector 320a for the unmapped proprietary code 302 in the cold start. Initially, attributes for the unmapped standard code 332 are targeted. The attributes include a “Names” attribute group which includes Long Common Name 334 (FIG. 3C), Short Name 336, Related Names2 338, and the Six Axes of LOINC 340. The text from the data fields of the “Names” attribute group are aggregated and preprocessed to form aggregated and preprocessed text 342. The mapping engine 318 generates an embedding vector 344 for the aggregated and preprocessed text 342.

FIG. 3D illustrates computing similarity scores for the weighted embeddings from the warm start and the similarity scores for the embedding vectors from the cold start process. More particularly, FAISS is used to compute a similarity value 350a for the weighted embedding 324 for the target unmapped proprietary code 302 (FIG. 3A) and the weighted embedding 344 of the mapped standard code from the warm start. Similarly, FAISS is used to compute a similarity value 350b for the embedding vector 320 for the target unmapped proprietary code 302 (FIG. 3A) and the embedding vector for the unmapped standard code 332 (FIG. 3C) from the cold start. The resulting similarity values 350a, 350b are received by a selection engine 352.

The operations may include repeating the warm start for each mapped standard code and repeating the cold start for each unmapped standard code. Top “N” Candidate Standard Codes (mapped and/or unmapped) for the Target Unmapped Proprietary Code are presented for selection in a recommendation interface 354.

### 5. Recommendation Interface

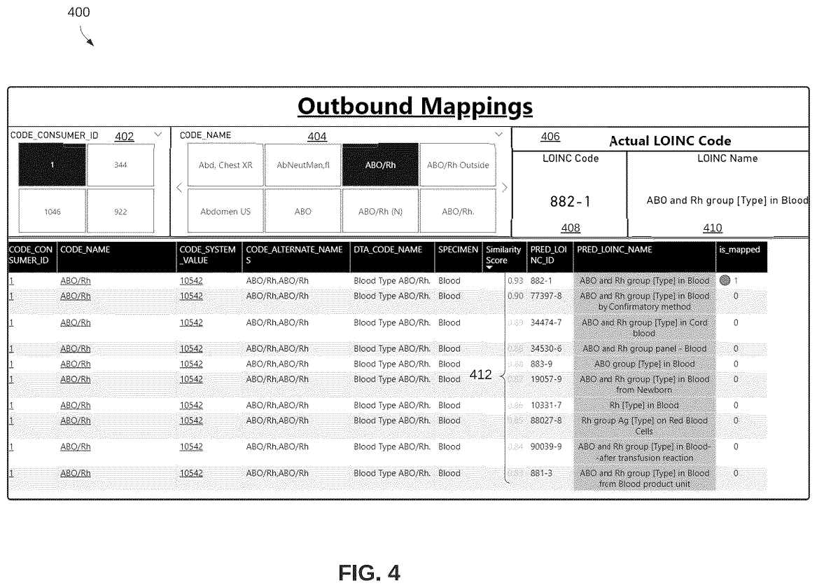

FIG. 4 illustrates an example of a recommendation interface 400 in accordance with one or more embodiments. The recommendation interface 400 may display information in a table format for easy viewing.

The interface 400 provides indication of a code consumer ID 402, a code name 404, a standard code name 406, a standard code identifier 408, and a standard code description 410. Candidate standard codes, provided in a table that includes Code Consumer ID, Code Name, Code System Value, Code Alternate Names, DTA Code Name, Specimen, Similarity Score, Pred LOINC ID, Pred LOINC Name, are mapped. The candidate mapped and unmapped standard codes are presented in ranked order based on similarities scores 412. Although ten (10) candidate standard codes are shown, it is envisioned that more or less than ten (10) candidate standard codes may be presented.

### 6. Hardware Overview

According to one embodiment, the techniques described herein are implemented by one or more special-purpose computing devices. The special-purpose computing devices may be hard-wired to perform the techniques, or may include digital electronic devices such as one or more application-specific integrated circuits (ASICs), field programmable gate arrays (FPGAs), or network processing units (NPUs) that are persistently programmed to perform the techniques, or may include one or more general purpose hardware processors programmed to perform the techniques pursuant to program instructions in firmware, memory, other storage, or a combination. Such special-purpose computing devices may also combine custom hard-wired logic, ASICs, FPGAs, or NPUs with custom programming to accomplish the techniques. The special-purpose computing devices may be desktop computer systems, portable computer systems, handheld devices, networking devices or any other device that incorporates hard-wired and/or program logic to implement the techniques.

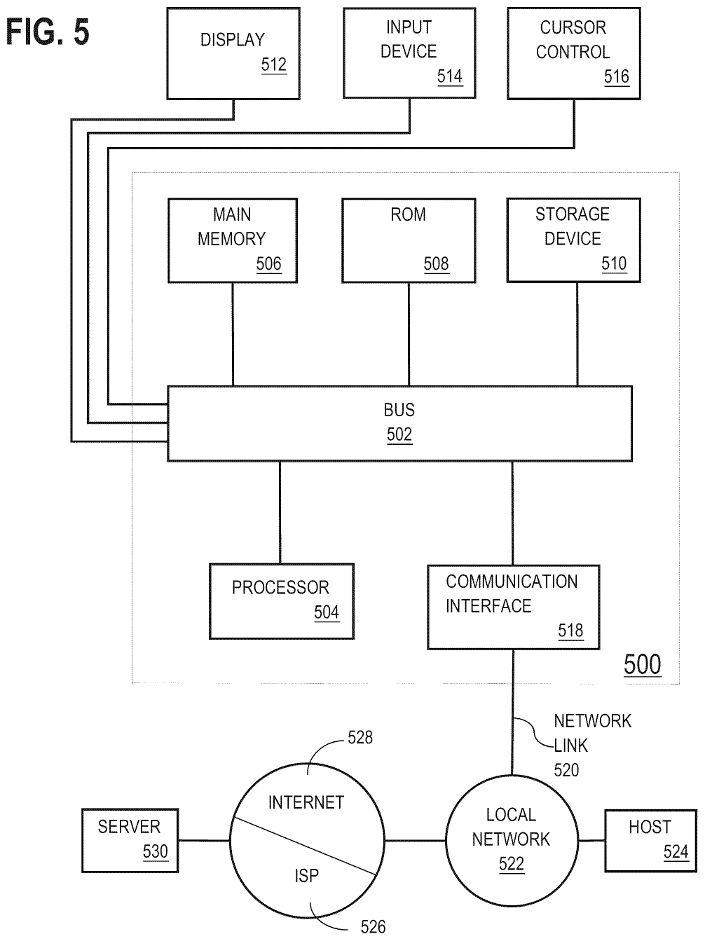

For example, FIG. 5 is a block diagram that illustrates a computer system 500 upon which an embodiment of the disclosure may be implemented. Computer system 500 includes a bus 502 or other communication mechanism for communicating information, and a hardware processor 504 coupled with bus 502 for processing information. Hardware processor 504 may be, for example, a general purpose microprocessor.

Computer system 500 also includes a main memory 506, such as a random access memory (RAM) or other dynamic storage device, coupled to bus 502 for storing information and instructions to be executed by processor 504. Main memory 506 also may be used for storing temporary variables or other intermediate information during execution of instructions to be executed by processor 504. Such instructions, when stored in non-transitory storage media accessible to processor 504, render computer system 500 into a special-purpose machine that is customized to perform the operations specified in the instructions.

Computer system 500 further includes a read only memory (ROM) 508 or other static storage device coupled to bus 502 for storing static information and instructions for processor 504. A storage device 510, such as a magnetic disk, optical disk, or a Solid State Drive (SSD) is provided and coupled to bus 502 for storing information and instructions.

Computer system 500 may be coupled via bus 502 to a display 512, such as a cathode ray tube (CRT), for displaying information to a computer user. An input device 514, including alphanumeric and other keys, is coupled to bus 502 for communicating information and command selections to processor 504. Another type of user input device is cursor control 516, such as a mouse, a trackball, or cursor direction keys for communicating direction information and command selections to processor 504 and for controlling cursor movement on display 512. This input device typically has two degrees of freedom in two axes, a first axis (e.g., x) and a second axis (e.g., y), that allows the device to specify positions in a plane.

Computer system 500 may implement the techniques described herein using customized hard-wired logic, one or more ASICs or FPGAs, firmware and/or program logic which in combination with the computer system causes or programs computer system 500 to be a special-purpose machine. According to one embodiment, the techniques herein are performed by computer system 500 in response to processor 504 executing one or more sequences of one or more instructions contained in main memory 506. Such instructions may be read into main memory 506 from another storage medium, such as storage device 510. Execution of the sequences of instructions contained in main memory 506 causes processor 504 to perform the process steps described herein. In alternative embodiments, hard-wired circuitry may be used in place of or in combination with software instructions.

The term “storage media” as used herein refers to any non-transitory media that store data and/or instructions that cause a machine to operate in a specific fashion. Such storage media may comprise non-volatile media and/or volatile media. Non-volatile media includes, for example, optical or magnetic disks, such as storage device 510. Volatile media includes dynamic memory, such as main memory 506. Common forms of storage media include, for example, a floppy disk, a flexible disk, hard disk, solid state drive, magnetic tape, or any other magnetic data storage medium, a CD-ROM, any other optical data storage medium, any physical medium with patterns of holes, a RAM, a PROM, and EPROM, a FLASH-EPROM, NVRAM, any other memory chip or cartridge, content-addressable memory (CAM), and ternary content-addressable memory (TCAM).

Storage media is distinct from but may be used in conjunction with transmission media. Transmission media participates in transferring information between storage media. For example, transmission media includes coaxial cables, copper wire and fiber optics, including the wires that comprise bus 502. Transmission media can also take the form of acoustic or light waves, such as those generated during radio-wave and infra-red data communications.

Various forms of media may be involved in carrying one or more sequences of one or more instructions to processor 504 for execution. For example, the instructions may initially be carried on a magnetic disk or solid state drive of a remote computer. The remote computer can load the instructions into its dynamic memory and send the instructions over a telephone line using a modem. A modem local to computer system 500 can receive the data on the telephone line and use an infra-red transmitter to convert the data to an infra-red signal. An infra-red detector can receive the data carried in the infra-red signal and appropriate circuitry can place the data on bus 502. Bus 502 carries the data to main memory 506, from which processor 504 retrieves and executes the instructions. The instructions received by main memory 506 may optionally be stored on storage device 510 either before or after execution by processor 504.

Computer system 500 also includes a communication interface 518 coupled to bus 502. Communication interface 518 provides a two-way data communication coupling to a network link 520 that is connected to a local network 522. For example, communication interface 518 may be an integrated services digital network (ISDN) card, cable modem, satellite modem, or a modem to provide a data communication connection to a corresponding type of telephone line. As another example, communication interface 518 may be a local area network (LAN) card to provide a data communication connection to a compatible LAN. Wireless links may also be implemented. In any such implementation, communication interface 518 sends and receives electrical, electromagnetic or optical signals that carry digital data streams representing various types of information.

Network link 520 typically provides data communication through one or more networks to other data devices. For example, network link 520 may provide a connection through local network 522 to a host computer 524 or to data equipment operated by an Internet Service Provider (ISP) 526. ISP 526 in turn provides data communication services through the world wide packet data communication network now commonly referred to as the “Internet” 528. Local network 522 and Internet 528 both use electrical, electromagnetic or optical signals that carry digital data streams. The signals through the various networks and the signals on network link 520 and through communication interface 518, which carry the digital data to and from computer system 500, are example forms of transmission media.

Computer system 500 can send messages and receive data, including program code, through the network(s), network link 520 and communication interface 518. In the Internet example, a server 530 might transmit a requested code for an application program through Internet 528, ISP 526, local network 522 and communication interface 518.

The received code may be executed by processor 504 as it is received, and/or stored in storage device 510, or other non-volatile storage for later execution.

### 7. Miscellaneous; Extensions

Unless otherwise defined, all terms (including technical and scientific terms) are to be given their ordinary and customary meaning to a person of ordinary skill in the art, and are not to be limited to a special or customized meaning unless expressly so defined herein.

This application may include references to certain trademarks. Although the use of trademarks is permissible in patent applications, the proprietary nature of the marks should be respected and every effort made to prevent their use in any manner which might adversely affect their validity as trademarks.

Embodiments are directed to a system with one or more devices that include a hardware processor and that are configured to perform any of the operations described herein and/or recited in any of the claims below.

In an embodiment, one or more non-transitory computer readable storage media comprises instructions which, when executed by one or more hardware processors, cause performance of any of the operations described herein and/or recited in any of the claims.

In an embodiment, a method comprises operations described herein and/or recited in any of the claims, the method being executed by at least one device including a hardware processor.

Any combination of the features and functionalities described herein may be used in accordance with one or more embodiments. In the foregoing specification, embodiments have been described with reference to numerous specific details that may vary from implementation to implementation. The specification and drawings are, accordingly, to be regarded in an illustrative rather than a restrictive sense. The sole and exclusive indicator of the scope of the disclosure, and what is intended by the applicants to be the scope of the disclosure, is the literal and equivalent scope of the set of claims that issue from this application, in the specific form in which such claims issue, including any subsequent correction.

What is claimed is:

1. One or more non-transitory computer readable media comprising instructions which, when executed by one or more hardware processors, cause performance of operations comprising:
generating a target vector embedding for a target unmapped proprietary code at least by:
applying a vector embedding function to a dataset of the target unmapped proprietary code to generate a target set of one or more vector embeddings;
identifying a plurality of mapped standard codes mapped to one or more proprietary codes;
generating a first plurality of vector embeddings corresponding to the plurality of mapped standard codes, wherein generating the first plurality of vector embeddings comprises:
generating a first vector embedding for a first mapped standard code of the plurality of mapped standard codes at least by:
applying a vector embedding function to a dataset of one or more proprietary codes mapped to the first mapped standard code;
computing a similarity measure for the target vector embedding and each of the first plurality of vector embeddings corresponding to the plurality of mapped standard codes to generate a first plurality of similarity values, the first plurality of similarity values comprise:
a first similarity measure for the target vector embedding and the first vector embedding of the first mapped standard code;
identifying a plurality of unmapped standard codes for mapping to the target unmapped proprietary code;
generating a second plurality of vector embeddings corresponding to the plurality of unmapped standard codes, wherein generating the second plurality of vector embeddings comprises:
generating a second vector embedding for a first unmapped standard code of the plurality of unmapped standard codes at least by:
applying a vector embedding function to a dataset of the first unmapped standard code;
computing a similarity measure for the target vector embedding and each of the second plurality of vector embeddings corresponding to the unmapped standard codes to generate a second plurality of similarity values, the second plurality of similarity values comprise:
a second similarity measure for the target vector embedding and the second vector embedding for the first unmapped standard code;
based on the first plurality of similarity values and the second plurality of similarity values, identifying a candidate standard code, the candidate standard code being selected from a combined set of standard codes comprising the plurality of mapped standard codes and the plurality of unmapped standard codes; and
presenting the candidate standard code for mapping to the target unmapped proprietary code.

2. The non-transitory media of claim 1, wherein generating a first vector embedding for a first mapped standard code of the plurality of mapped standard codes further comprises:
applying a vector embedding function to a dataset of the first mapped standard code.

3. The non-transitory media of claim 1, wherein the candidate standard code comprises the first unmapped standard code, wherein the candidate standard code is mapped to the target unmapped proprietary code, wherein the operations further comprise:
generating a third vector embedding for the candidate standard code based on a dataset mapped to the candidate standard code and a dataset mapped to the target unmapped proprietary code; and
using the third vector embedding to map one or more additional unmapped proprietary codes.

4. The non-transitory media of claim 1, wherein generating the first vector embedding for a first mapped standard code of the plurality of mapped standard codes comprises:
applying the vector embedding function to the dataset of the one or more proprietary codes mapped to the first mapped standard code to generate a first set of vector embeddings for the first mapped standard code; and
generating the first vector embedding based on the first set of vector embeddings for the first mapped standard code.

5. The non-transitory media of claim 1, wherein generating the first vector embedding for a first unmapped standard code of the plurality of unmapped standard codes comprises:
applying the vector embedding function to the dataset of the first unmapped standard code to generate a first set of vector embeddings for the first unmapped standard code; and
generating the first vector embedding based on the first set of vector embeddings for the first unmapped standard.

6. The non-transitory media of claim 1, wherein generating the first plurality of vector embeddings for the mapped standard codes further comprises:
generating a third vector embedding for a second mapped standard code of the plurality of mapped standard codes at least by:
applying the vector embedding function to a dataset of one or more proprietary codes mapped to the second mapped standard code;
wherein the first plurality of similarity values further comprise:
a third similarity measure for the target vector embedding and the second vector embedding for the second mapped standard code;
wherein the operations further comprise:
based at least on the third similarity measure, refraining from presenting the second mapped standard code as any candidate mapped standard code for mapping to the target unmapped proprietary code.

7. The non-transitory media of claim 1, wherein generating the second plurality of vector embeddings for the unmapped standard codes further comprises:
generating a third vector embedding for a second unmapped standard code of the plurality of unmapped standard codes at least by:
applying the vector embedding function to a dataset of the second unmapped standard code;
wherein the second plurality of similarity values further comprise:
a third similarity measure for the target vector embedding and the second vector embedding for a second unmapped standard code;
wherein the operations further comprise:
based at least on the third similarity measure, refraining from presenting the second unmapped standard code as any candidate unmapped standard code for mapping to the target unmapped proprietary code.

8. The non-transitory media of claim 1, wherein the first and second similarity measures are computed using Facebook AI Similarity Search (FAISS) combined with Hierarchical Navigable Small World (HNSW) as an indexing approach.

9. The non-transitory media of claim 1, wherein the operations further comprise:
identifying “N” highest similarity values of the first and second plurality of similarity values; and
presenting the mapped and unmapped standard codes, mapped to vector embeddings that correspond to the “N” highest similarity values, as the candidate standard codes for mapping to the target unmapped proprietary code.

10. The non-transitory media of claim 1, wherein applying the vector embedding function to the dataset of mapped or unmapped standard codes comprises using Self-Alignment Pretraining for Biomedical Entity Representations (SAPBERT) word embedding technique.

11. A method comprising:
generating a target vector embedding for a target unmapped proprietary code at least by:
applying a vector embedding function to a dataset of the target unmapped proprietary code to generate a target set of one or more vector embeddings;
identifying a plurality of mapped standard codes mapped to one or more proprietary codes;
generating a first plurality of vector embeddings corresponding to the plurality of mapped standard codes, wherein generating the first plurality of vector embeddings comprises:
generating a first vector embedding for a first mapped standard code of the plurality of mapped standard codes at least by:
applying a vector embedding function to a dataset of one or more proprietary codes mapped to the first mapped standard code;
computing a similarity measure for the target vector embedding and each of the first plurality of vector embeddings corresponding to the plurality of mapped standard codes to generate a first plurality of similarity values, the first plurality of similarity values comprise:
a first similarity measure for the target vector embedding and the first vector embedding of the first mapped standard code;
identifying a plurality of unmapped standard codes for mapping to the target unmapped proprietary code;
generating a second plurality of vector embeddings corresponding to the plurality of unmapped standard codes, wherein generating the second plurality of vector embeddings comprises:
generating a second vector embedding for a first unmapped standard code of the plurality of unmapped standard codes at least by:
applying a vector embedding function to a dataset of the first unmapped standard code;
computing a similarity measure for the target vector embedding and each of the second plurality of vector embeddings corresponding to the unmapped standard codes to generate a second plurality of similarity values, the second plurality of similarity values comprise:
a second similarity measure for the target vector embedding and the second vector embedding for the first unmapped standard code;
based on the first plurality of similarity values and the second plurality of similarity values, identifying a candidate standard code, the candidate standard code being selected from a combined set of standard codes comprising the plurality of mapped standard codes and the plurality of unmapped standard codes; and
presenting the candidate standard code for mapping to the target unmapped proprietary code, wherein the method is performed by at least one device including a hardware processor.

12. The method of claim 11, wherein generating a first vector embedding for a first mapped standard code of the plurality of mapped standard codes further comprises:
applying a vector embedding function to a dataset of the first mapped standard code.

13. The method of claim 11, wherein the candidate standard code comprises the first unmapped standard code, wherein the candidate standard code is mapped to the target unmapped proprietary code, wherein the operations further comprise:
generating a third vector embedding for the candidate standard code based on a dataset mapped to the candidate standard code and a dataset mapped to the target unmapped proprietary code; and
using the third vector embedding to map one or more additional unmapped proprietary codes.

14. The method of claim 11, wherein generating the first vector embedding for a first mapped standard code of the plurality of mapped standard codes comprises:
applying the vector embedding function to the dataset of the one or more proprietary codes mapped to the first mapped standard code to generate a first set of vector embeddings for the first mapped standard code; and
generating the first vector embedding based on the first set of vector embeddings for the first mapped standard code.

15. The method of claim 11, wherein generating the first vector embedding for a first unmapped standard code of the plurality of unmapped standard codes comprises:
applying the vector embedding function to the dataset of the first unmapped standard code to generate a first set of vector embeddings for the first unmapped standard code; and
generating the first vector embedding based on the first set of vector embeddings for the first unmapped standard.

16. The method of claim 11, wherein generating the first plurality of vector embeddings for the mapped standard codes further comprises:
generating a third vector embedding for a second mapped standard code of the plurality of mapped standard codes at least by:
applying the vector embedding function to a dataset of one or more proprietary codes mapped to the second mapped standard code;
wherein the first plurality of similarity values further comprise:
a third similarity measure for the target vector embedding and the second vector embedding for the second mapped standard code;
wherein the operations further comprise:
based at least on the third similarity measure, refraining from presenting the second mapped standard code as any candidate mapped standard code for mapping to the target unmapped proprietary code.

17. The method of claim 11, wherein generating the second plurality of vector embeddings for the unmapped standard codes further comprises:
generating a third vector embedding for a second unmapped standard code of the plurality of unmapped standard codes at least by:
applying the vector embedding function to a dataset of the second unmapped standard code;
wherein the second plurality of similarity values further comprise:
a third similarity measure for the target vector embedding and the second vector embedding for a second unmapped standard code;
wherein the operations further comprise:
based at least on the third similarity measure, refraining from presenting the second unmapped standard code as any candidate unmapped standard code for mapping to the target unmapped proprietary code.

18. The method of claim 11, wherein the first and second similarity measures are computed using Facebook AI Similarity Search (FAISS) combined with Hierarchical Navigable Small World (HNSW) as an indexing approach.

19. The method of claim 11, wherein the operations further comprise:
identifying “N” highest similarity values of the first and second plurality of similarity values; and
presenting the mapped and unmapped standard codes, mapped to vector embeddings that correspond to the “N” highest similarity values, as the candidate standard codes for mapping to the target unmapped proprietary code.

20. A system comprising:
at least one device including a hardware processor;
the system being configured to perform operations comprising:
generating a target vector embedding for a target unmapped proprietary code at least by:
applying a vector embedding function to a dataset of the target unmapped proprietary code to generate a target set of one or more vector embeddings;
identifying a plurality of mapped standard codes mapped to one or more proprietary codes;
generating a first plurality of vector embeddings corresponding to the plurality of mapped standard codes, wherein generating the first plurality of vector embeddings comprises:
generating a first vector embedding for a first mapped standard code of the plurality of mapped standard codes at least by:
applying a vector embedding function to a dataset of one or more proprietary codes mapped to the first mapped standard code;
computing a similarity measure for the target vector embedding and each of the first plurality of vector embeddings corresponding to the plurality of mapped standard codes to generate a first plurality of similarity values, the first plurality of similarity values comprise:
a first similarity measure for the target vector embedding and the first vector embedding of the first mapped standard code;
identifying a plurality of unmapped standard codes for mapping to the target unmapped proprietary code;
generating a second plurality of vector embeddings corresponding to the plurality of unmapped standard codes, wherein generating the second plurality of vector embeddings comprises:
generating a second vector embedding for a first unmapped standard code of the plurality of unmapped standard codes at least by:
applying a vector embedding function to a dataset of the first unmapped standard code;
computing a similarity measure for the target vector embedding and each of the second plurality of vector embeddings corresponding to the unmapped standard codes to generate a second plurality of similarity values, the second plurality of similarity values comprise:
a second similarity measure for the target vector embedding and the second vector embedding for the first unmapped standard code;
based on the first plurality of similarity values and the second plurality of similarity values, identifying a candidate standard code, the candidate standard code being selected from a combined set of standard codes comprising the plurality of mapped standard codes and the plurality of unmapped standard codes; and
presenting the candidate standard code for mapping to the target unmapped proprietary code.
